SEQUENCE LISTING (8 sequences)

SEQ ID NO: 1 - DNA, 4641 bp, synthetic construct
  source 1..4641: mol_type = other DNA
atgaacaaatcttcccgtaccctgctctctctgggcctgctgagcgcggccatgttcggcgtttcgcaacaggcgaatgcccacggttatgtcgaatcaccggccagccgcgcctatcagtgcaaactgcagctcaacacccagtgcggcagcgtgcagtatgaaccgcagagcgtcgaaggcctgaaaggcttcccgcaggccggtccggctgacggccatatcgccagcgccgacaaatccaccttcttcgaactggatcagcaaacgccgacgcgctggaacaagctcaacctgaaaaccggccccaactcctttacctggaagctgaccgcgcgtcacagcactaccagctggcgctatttcatcactaagccgaactgggacgcttcgcaaccgctgacccgcgcctcctttgatctgacgccgttctgccagttcaacgacggcggcgccattcctgccgcgcaggtgacccaccagtgcaacataccggcagatcgcagcggttcgcacgtgatccttgccgtgtgggatatagccgacaccgccaacgccttctaccaggcgatcgatgtcaacctgagcaaataattaggcgatgagctgccacagggtgaaggcggcgtccggcgtccagccggcgttggaagtgtgcgcctggatacaggtgtatttcttgcctttataactcaccacgtcgccggctttgtagctgtggttgttctgccactcgggcgcccccggatcgggcggcgtgccgtcgttggcggttttgaccgccagcgcactgctcggcagcgaggtattgccctggctgtcggtcgcgataacgaagtagctgtactgggtggccggcgtcaggccgctgtcggccagtgacagggcggcggtctggccgatcgggttgccgttgcggtagacggtgtaactggcgatcggtgaggcgccggtggcggcagcccagctcagtttcagcgaggtggcacccagatcggcgaccgtcagcgccgtcggcgcattaggctttcccggcggcggggtgacgccgccctgaatcagcggcgcgtagcgggttttgaattcccagttgtaggcgacgccggctttgctcttgccattgtcccagttgattgaccaggtcatcaggcccttgatcgacaggttcttggcgtcgagacgcgagaaggcgttatacaccgcctgtttgtcgatcacgtagccggtggcggcggcgtcgttgttgctcggcaggccgatgacgaatttcgccgccgggatcttcgcatagccgcgggtgccggtaaccaggctttccgtcaggtagtagaggaagtcctctttcatggcgtcgttattctgcgtgatccaggcattgagttcatccacccaaataccgtcgccgccctggttataatattgcggcgcgataaagtcgtaatagccttcgagggcgttgatataatccaggtaggtgccgttggtgcgcaaatacgggaattccggcgccatgctgataataaagttcttcccctgcgcagcgtaatagtcttttacttttttcaacgccgcaggcaagacagttttattattggctgcaccgatcgccgcctgttccagatcgatatccaggccgtcgaagccatagacttccaccaggcgaataatctcgtctttcagcttatcttcgtcgccggtcttcagttcgatatgcgcgtctgcgccgccgagggagatcagcaccgcgcggccctggctgttcagcacgcccacctgtcggcggaactcggcgtcggacaggttgtaaggtttgaaggttgggatgccctggcctttcataaaggcgacggccaccacattgtattcggcgggaatgtcggtcaggttcatattggcgaactggccttgctgatagccgtcactggcgccggcggcccagttgtgccaaaaacccatcaggatttttttattggcgatagtcggcataatggctgcgtcatcggcggcggcattaatcatgttatttgtgctcatatggatttgactgcaacgcagcctactaaagatgcccggtttcatggcggttctgaatatgcaaaagagatttttttcagatcgattgaatctgggtatgtcaactttcaatgcgtttataacaagaactacgaactcgatgaggctattgttagtgcttgtgtcaaaaatgatatactgctttatccagtggaaaataatgaaggtttgtataaatacattgaagctgttgagccacgggttttttattctgccatgccttatcagttgcatgagttgcctactgcacatactcatttcataatgacagtccacggtttaagggagattgagtgcccatatgataatgttcagtgggcatatacggcgggggttgttagtaggctaaaattgtttattaaggctttcgttttaaaggattacacaataaaaaaatacatttctatttttgaacgattgctaactaaaaaaaatataacagtgattacggtttctgagcatacaaaatactcgattctttccaattttagcaagtttgaaccaagtaatatccatgtgtttcccgcaccatgtccattcggaaatgacgaaccagtgttatctccagagttgatgtctatgcagttgaagggaaggggctttttcttgctagtcagtgctaatcgatggatgaaaaacaatcaccgtgccttgctcgcactatctaatttttatgataaaaacccagaatgtgaaatcataacgatagtgactggtgcaaaaaaaccacctaatttcttgaagggaaataatagatttaaatttttggattatatagatacttcatcattgaattggctctataaaaatgcatatagttttatctatccgagtcttaatgagggttatggttaccctcctttacaagcgatgcactatggcacgcctgttttggcctcgtctgtctcaagtattcctgaggtgtgcaaagacaatgttatttattttgatcccagaaatcctcgtgagatcgaaaaccgtatttttcaactttacttggattccgagcgttacgagagtttaaggaaaaaaggattgcaacatagcatgcatgtgcagggtgaacaagagttgaagatgaatgcattgatttcaatgatatttaataagacttatagcaatggctcgatgaatgatgacgtaaaaaaaagcacgattactggcctgctttggagtgcggcagaacgtttttctgttcaaggatttcaatttgtaataagtattattcttgcgagaattatatcacctgcggaatttgggattgttggaataattttagtttttgttcttatatctgatatcataataaatagtggtttttcacaggcgctaatccaaaagaaagatagggataatattgatctttcaacagtgttctattttaatgtgattataggtgtcgttttatacggtgtgctattttttttgtcgcctaccatagctagctattacgagaagccagagcttgaacatgtgctaaagatgatatctatagtaatcatgattaagccattatatctggttcatatgactttgctatcaataaggttggattttaagttacgaactaaaataaatttcattagcgctttcctgagtggcggtatcgcagtttttttagcaagtaaaggttttagtgtttatgcactcgtctggcaacaaatagccaacaattttctcattgtcttgctgtctttttttcttattaaatggattcctgggttggttttttcattcagttcacttgcaagtctctttagttttggttccaagttgttgatagcaggtcttgttaggagcgttgttgataatggatatgctatattgatagggaaatacttgtcgattaaagatgtagggctatatactcaagggagaaatatacccgatcttatatcaatgaatttattcactatattgcaaaatgttttctttcctgttatgtcgaatctacagaatgatcgtgagtcattacttaaattttatcatcgtggagttgaaggcgttgcttttctaattgttcccgtgatgattggcttgatgatgatatcggagccattcgtaagatattttctttcggagcagtggatgggcgccatcgttgtaatgcagtgggttgcattatcacgattaattattccaattagtgcactgaactgcagtttgataaattctataggacgttcggacgcttatttaaaggttgatttagctaaactacctattaccgttgccgcgctattagttactgttcctttcggattgaaggttgttgttattggtaatgctttagtctcctttatttgtttttttataaacgcttattatcctgggaagtggtttggttatggtgcaattaaacaaataaaaaccatgtttcctattctgattagtgctttgataatggcagcctctatatattttatccattttgaaaatggactgttagagatttttgttaaaattaccgtgggtgtaattgtttattatatatctgcgctaatcttgaaggttaaaatttgtcaagaaattaatcatgtttttttaaaagctgtcttcaaaagataa

SEQ ID NO: 2 - protein, 197 aa, Serratia sp.
MNKSSRTLLSLGLLSAAMFGVSQQANAHGYVESPASRAYQCKLQLNTQCGSVQYEPQSVEGLKGFPQAGPADGHIASADKSTFFELDQQTPTRWNKLNLKTGPNSFTWKLTARHSTTSWRYFITKPNWDASQPLTRASFDLTPFCQFNDGGAIPAAQVTHQCNIPADRSGSHVILAVWDIADTANAFYQAIDVNLSK

SEQ ID NO: 3 - protein, 95 aa, Serratia sp.
LGDELPQGEGGVRRPAGVGSVRLDTGVFLAFITHHVAGFVAVVVLPLGRPRIGRRAVVGGFDRQRTARQRGIALAVGRDNEVAVLGGRRQAAVGQ

SEQ ID NO: 4 - protein, 20 aa, Serratia sp.
QGGGLADRVAVAVDGVTGDR

SEQ ID NO: 5 - protein, 63 aa, Serratia sp.
GAGGGSPAQFQRGGTQIGDRQRRRRIRLSRRRGDAALNQRRVAGFEFPVVGDAGFALAIVPVD

SEQ ID NO: 6 - protein, 299 aa, Serratia sp.
PGHQALDRQVLGVETREGVIHRLFVDHVAGGGGVVVARQADDEFRRRDLRIAAGAGNQAFRQVVEEVLFHGVVILRDPGIEFIHPNTVAALVIILRRDKVVIAFEGVDIIQVGAVGAQIREFRRHADNKVLPLRSVIVFYFFQRRRQDSFIIGCTDRRLFQIDIQAVEAIDFHQANNLVFQLIFVAGLQFDMRVCAAEGDQHRAALAVQHAHLSAELGVGQVVRFEGWDALAFHKGDGHHIVFGGNVGQVHIGELALLIAVTGAGGPVVPKTHQDFFIGDSRHNGCVIGGGINHVICAH

SEQ ID NO: 7 - protein, 389 aa, Serratia sp.
MDLTATQPTKDARFHGGSEYAKEIFFRSIESGYVNFQCVYNKNYELDEAIVSACVKNDILLYPVENNEGLYKYIEAVEPRVFYSAMPYQLHELPTAHTHFIMTVHGLREIECPYDNVQWAYTAGVVSRLKLFIKAFVLKDYTIKKYISIFERLLTKKNITVITVSEHTKYSILSNFSKFEPSNIHVFPAPCPFGNDEPVLSPELMSMQLKGRGFFLLVSANRWMKNNHRALLALSNFYDKNPECEIITIVTGAKKPPNFLKGNNRFKFLDYIDTSSLNWLYKNAYSFIYPSLNEGYGYPPLQAMHYGTPVLASSVSSIPEVCKDNVIYFDPRNPREIENRIFQLYLDSERYESLRKKGLQHSMHVQGEQELKMNALISMIFNKTYSNGS

SEQ ID NO: 8 - protein, 479 aa, Serratia sp.
MNDDVKKSTITGLLWSAAERFSVQGFQFVISIILARIISPAEFGIVGIILVFVLISDIIINSGFSQALIQKKDRDNIDLSTVFYFNVIIGVVLYGVLFFLSPTIASYYEKPELEHVLKMISIVIMIKPLYLVHMTLLSIRLDFKLRTKINFISAFLSGGIAVFLASKGFSVYALVWQQIANNFLIVLLSFFLIKWIPGLVFSFSSLASLFSFGSKLLIAGLVRSVVDNGYAILIGKYLSIKDVGLYTQGRNIPDLISMNLFTILQNVFFPVMSNLQNDRESLLKFYHRGVEGVAFLIVPVMIGLMMISEPFVRYFLSEQWMGAIVVMQWVALSRLIIPISALNCSLINSIGRSDAYLKVDLAKLPITVAALLVTVPFGLKVVVIGNALVSFICFFINAYYPGKWFGYGAIKQIKTMFPILISALIMAASIYFIHFENGLLEIFVKITVGVIVYYISALILKVKICQEINHVFLKAVFKR

(19) United States
(12) Patent Application Publication
(10) Pub. No.: US 2025/0059551 A1
(43) Pub. Date: Feb. 20, 2025

(54) CORN ENDOTYPE-DERIVED POLYPEPTIDES CAPABLE OF METABOLIZING FUMONISIN

(71) Applicant: The Texas A&M University System, College Station, TX (US)

(72) Inventor: Ken Obasa, Amarillo, TX (US)

(21) Appl. No.: 18/804,525

(22) Filed: Aug. 14, 2024

Related U.S. Application Data
(60) Provisional application No. 63/519,424, filed on Aug. 14, 2023.

(51) Int. Cl. C12N 15/82; A01N 63/20; A01P 3/00; C12N 9/10; C12N 9/22; C12N 9/42; C12Q 1/6844; C12Q 1/6895

(52) U.S. Cl. CPC C12N 15/8243; A01N 63/20; A01P 3/00; C12N 9/1048; C12N 9/22; C12N 9/2442; C12N 15/8201; C12Q 1/6844; C12Q 1/6895; C12N 2310/20; C12Q 2600/13; C12Y 302/01014

(57) ABSTRACT

Polynucleotides and polypeptides conferring the ability to detoxify or metabolize the mycotoxin fumonisin are provided, as are constructs, compositions, and plants comprising the polynucleotides and polypeptides. Also provided are plants having the ability to detoxify fumonisin, methods of conferring fumonisin detoxification, methods of controlling fumonisin contamination, and kits and methods for identification of plants having the ability to detoxify fumonisin.

### CROSS REFERENCE TO RELATED APPLICATIONS

This application claims priority under 35 U.S.C. § 119 to provisional patent application U.S. Ser. No. 63/519,424, filed Aug. 14, 2023. The provisional patent application is herein incorporated by reference in its entirety, including without limitation, the specification, claims, and abstract, as well as any figures, tables, appendices, or drawings thereof.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Aug. 13, 2024 is named “P14480US01_SequenceListing.xml” and is 14,145 bytes in size.

### TECHNICAL FIELD

The present disclosure relates generally to compositions and methods for conferring the ability to detoxify fumonisin in plants, including polynucleotide constructs and polypeptides.

### BACKGROUND

Fumonisin contamination of field corn remains an economically important issue across many corn producing regions of the United States and globally. The conditions that favor ear infection by fungal pathogens, including fumonisin-producing Fusarium species, are also projected by climate prediction models to become exacerbated in the coming years. This represents not only a threat to the production of a staple food crop but will impact the quality as well, with potential adverse implications for humans and animals that consume mycotoxin contaminated food and feed products.

There is currently no corn hybrid having fumonisin resistant traits. Additionally, available products for mitigating fumonisin contamination in corn are applicable only to corn products and are not designed for incorporation directly into corn plants.

Thus, there is a need in the art for the development of a technology that offers the potential for incorporation directly into corn plants for expression in corn ears. Among other benefits, this would enable the mitigation of fumonisin at the source, eliminating the risks and downstream costs associated with managing the potential adverse health effects of fumonisin.

### SUMMARY

Plant varieties having an ability to detoxify fumonisin are provided. In some embodiments, a plant, a plant part, or a seed of said variety comprises a heterologous polynucleotide construct encoding a chitin-binding polypeptide, a chitinase polypeptide, a glycosyltransferase polypeptide, and a lipopolysaccharide biosynthesis polypeptide, wherein the chitin-binding polypeptide, chitinase polypeptide, glycosyltransferase polypeptide, and lipopolysaccharide biosynthesis polypeptide are Serratia sp. polypeptides.

Methods for producing a plant, plant part, or plant cell with an ability to detoxify fumonisin are also provided. In some embodiments, the method comprises modifying the plant to comprise a heterologous polynucleotide construct encoding a chitin-binding polypeptide, a chitinase polypeptide, a glycosyltransferase polypeptide, and a lipopolysaccharide biosynthesis polypeptide, wherein the chitin-binding polypeptide, chitinase polypeptide, glycosyltransferase polypeptide, and lipopolysaccharide biosynthesis polypeptide are Serratia sp. polypeptides.

Polynucleotide constructs that confer fumonisin detoxification in plants are also provided. In some embodiments, the construct encodes a chitin-binding polypeptide, a chitinase polypeptide, a glycosyltransferase polypeptide, and a lipopolysaccharide biosynthesis polypeptide, wherein the chitin-binding polypeptide, chitinase polypeptide, glycosyltransferase polypeptide, and lipopolysaccharide biosynthesis polypeptide are Serratia sp. polypeptides. In some embodiments, the polynucleotide has at least 70%, at least 80%, at least 90%, at least 95%, at least 98%, at least 99%, or 100% sequence identity to SEQ ID NO: 1. In some embodiments, the construct encodes a chitin-binding polypeptide, wherein the chitin-binding polypeptide comprises an amino acid sequence having at least 70%, at least 80%, at least 90%, at least 95%, at least 98%, at least 99%, or 100% sequence identity to SEQ ID NO: 2; a chitinase polypeptide, wherein the chitinase polypeptide comprises an amino acid sequence having at least 70%, at least 80%, at least 90%, at least 95%, at least 98%, at least 99%, or 100% sequence identity to SEQ ID NO: 3, 4, 5, and/or 6; a glycosyltransferase polypeptide, wherein the glycosyltransferase polypeptide comprises an amino acid sequence having at least 70%, at least 80%, at least 90%, at least 95%, at least 98%, at least 99%, or 100% sequence identity to SEQ ID NO: 7; and a lipopolysaccharide biosynthesis polypeptide, wherein the lipopolysaccharide biosynthesis polypeptide comprises an amino acid sequence having at least 70%, at least 80%, at least 90%, at least 95%, at least 98%, at least 99%, or 100% sequence identity to SEQ ID NO: 8.

Methods for identifying a plant having an ability to detoxify fumonisin are also provided. In certain embodiments, the method comprises obtaining a nucleic acid sample from a plant suspected of having the ability to detoxify fumonisin; detecting in the sample a polynucleotide construct that confers fumonisin detoxification in plants encoding a chitin-binding polypeptide, a chitinase polypeptide, a glycosyltransferase polypeptide, and a lipopolysaccharide biosynthesis polypeptide; and determining that the plant has an increased resistance to fumonisin based on the presence of the polynucleotide. Kits for identifying a plant having the ability to detoxify fumonisin are also provided.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIGS. 1A-1B show corn sample selection and bacterial isolation as described in Example 1. FIG. 1A shows corn stalk sectioned and placed on growth media to isolate bacteria present within the stalk tissues. FIG. 1B shows plates having bacteria isolated from tissues of corn stalks that were sectioned and previously incubated on the plate.

FIGS. 2A-2B show corn inoculation experiments described in Example 2. FIG. 2A shows corn plants in the greenhouse with two replicate pots per treatment. FIG. 2B shows corn plants in the greenhouse with two replicate pots per treatment under supplemental LED lighting.

FIGS. 3A-3C illustrate the results of the corn inoculation experiments. FIG. 3A shows fumonisin levels measured in corn kernels following simultaneous inoculation of corn ears, separately, with mixtures of bacterial consortia and conidial suspension of a toxigenic Fusarium isolate, KC8-3. FIG. 3B shows fumonisin levels following inoculation of corn ears, separately, with bacterial consortia and conidial suspensions of a toxigenic Fusarium isolate KC8-3 in three different inoculation orders, under greenhouse conditions. FIG. 3C shows representative corn kernels for three inoculation sequences and their respective average visual quality ratings, based on a 1-10 scale. Simultaneous: Corn ears inoculated simultaneously with bacterial consortia and fungal inoculum; B+F: Corn ears inoculated with bacterial consortia 96 h prior to inoculation with fungus; F+B: Corn ears inoculated with fungal conidia 96 h prior to inoculation with bacterial consortia.

FIGS. 4A-4B show a map of an exemplary enzyme fusion protein comprising enzymes isolated from Serratia sp. B1072. The enzymes include chitin binding protein (SEQ ID NO: 2), chitinase (SEQ ID NOs: 3-6), glycosyltransferase family 4 protein (SEQ ID NO: 7), and lipopolysaccharide biosynthesis protein (SEQ ID NO: 7). The asterisks represent stop codons, with the chitinase having stop codons due to alternative splicing.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation; the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about”, as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

As used herein, the phrase “biological sample” refers to either intact or non-intact (e.g., milled seed or plant tissue, chopped plant tissue, lyophilized tissue) plant tissue. It may also be an extract comprising intact or non-intact seed or plant tissue. The biological sample can comprise flour, meal, syrup, oil, starch, and cereals manufactured in whole or in part to contain crop plant by-products. In certain embodiments, the biological sample is “non-regenerable” (i.e., incapable of being regenerated into a plant or plant part).

As used herein, the term “confer” refers to providing a characteristic or trait, such as the ability to detoxify fumonisin and/or other desirable traits to a plant.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the term “detoxify” or “fumonisin detoxication” refers to the ability of a plant to metabolize or degrade fumonisin into less toxic substances. “Fumonisin resistance” may be used interchangeably with fumonisin detoxification.

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule.

As used herein, the terms “elite,” “elite variety,” and “elite line” or “elite plant” refer to any line or plant that has resulted from breeding and/or gene editing and selection for one or more trait improvements (e.g., desirable agronomic performance (typically commercial production) or superior grain quality). Generally, individuals in a line have similar parentage and one or more similar traits. In some cases, an “elite line” or “elite variety” can be an agronomically or otherwise superior line or variety that has resulted from several or many cycles of breeding and selection for one or more trait improvements (e.g., superior agronomic performance or superior grain quality). An “elite inbred line” is an elite line that is an inbred, and that has been shown to be useful for producing sufficiently high yielding and agronomically fit hybrid varieties (an “elite hybrid variety”). Similarly, “elite germplasm” is a germplasm resulting from breeding and selection for desirable agronomic performance (typically commercial production). Such germplasm may be agronomically superior germplasm, derived from and/or capable of giving rise to a plant with superior agronomic performance, such as an existing or newly developed elite line of Brassica (e.g., pennycress). “Commercially elite,” “commercially elite plants,” “commercially elite seed,” “elite commercial,” “elite commercial plants,” “elite commercial seed,” or “elite commercial varieties” are elite varieties which can be used by growers to produce a profitable crop, which is purchased (e.g., as seed) for use by a grower to produce a crop, or for which a user of the crop pays for access to the seed.

As used herein, an “endogenous gene” or a “native copy” of a gene refers to a gene that originates from within a given organism, cell, tissue, genome, or chromosome. An “endogenous gene” or a “native copy” of a gene is a gene that was not previously modified by human action. Similarly, an “endogenous protein” refers to a protein encoded by an endogenous gene.

As used herein, “expression” means the production of a protein through the process of transcribing a DNA molecule into messenger RNA (mRNA) and translating the mRNA into polypeptide chains, which are ultimately folded into proteins.

Fusarium verticillioides, F. proliferatum, and F. subglutinans are the fungal pathogens that induce Fusarium ear mold (or ear rot) in maize and other cereal crops. The fungal pathogens are also referred to collectively herein as Fusarium.

“Fumonisin” as used herein refers to a group of mycotoxins produced by Fusarium. Fumonisin is a common contaminant of corn, corn products, and other cereals such as rice, wheat, barley, corn, rye, sorghum, soybeans, oats, and millet. Fumonisin refers to any one or more of all fumonisins, including fumonisins A, B, C, and P. Fumonisin B includes FB1, FB2, FB3, and FB4.

As used herein, the terms “include,” “includes,” and “including” are to be construed as at least having the features to which they refer while not excluding any additional unspecified features.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

As used herein, an “isolated” nucleic acid molecule is substantially separated away from other nucleic acid sequences with which the nucleic acid is normally associated, such as, from the chromosomal or extrachromosomal DNA of a cell in which the nucleic acid naturally occurs. The term also embraces nucleic acids that are biochemically purified so as to substantially remove contaminating nucleic acids and other cellular components.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript. Modified plants or seeds contain molecular changes in their genetic materials, including either genetic or epigenetic modifications. Typically, modified plants or seeds, or a parental or progenitor line thereof, have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In certain embodiments, a modified plant provided herein comprises no non-plant genetic material or sequences. In certain embodiments, a modified plant provided herein comprises no interspecies genetic material or sequences.

As used herein, “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A progeny plant can be from any filial generation, e.g., F1, F2, F3, F4, F5, F6, F7, etc. A plant cell is a biological cell of a plant, taken from a plant or derived through culture from a cell taken from a plant. Plant parts include, but are not limited to seeds microspores, pollen, anthers, silk, spike, ovules, ovaries, flowers, pods, cobs, embryos, stems, leaves, roots, and calli.

The term “polynucleotide” as used herein is a nucleic acid molecule comprising a plurality of polymerized nucleotides, e.g., at least about five consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, amplicon, primer, oligomer, element, target, and probe and in some embodiments is single-stranded.

The term “primer” as used herein encompasses any nucleic acid that is capable of priming the synthesis of a nascent nucleic acid in a template-dependent process, such as PCR. Typically, primers are oligonucleotides from 10 to 30 nucleotides in length, but longer sequences may be used. Primers may be provided in single or double-stranded form. Probes may be used as primers, but are designed to bind to the target DNA or RNA and need not be used in an amplification process.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Illustrative plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters that initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter that is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter that is active under most environmental conditions.

As used herein, “recombinant,” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if that nucleotide sequence has been removed from its natural context and cloned into any type of artificial nucleic acid vector. The term recombinant also can refer to an organism having a recombinant material, e.g., a plant that comprises a recombinant nucleic acid can be considered a recombinant plant.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory elements may include, but are not limited to, promoters, translation leader sequences, introns, and polyadenylation recognition sequences. Regulatory elements present on a recombinant DNA construct that is introduced into a cell can be endogenous to the cell, or they can be heterologous with respect to the cell. The terms “regulatory element” and “regulatory sequence” are used interchangeably herein.

A “sequence” means a sequential arrangement of nucleotides or amino acids. The boundaries of a protein-coding sequence may be determined by a translation start codon at the 5′-terminus and a translation stop codon at the 3′-terminus. In certain embodiments, a protein-coding molecule may comprise a DNA sequence encoding a protein sequence. In certain embodiments, a protein-coding molecule may comprise a RNA sequence encoding a protein sequence.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., Madison, Wis.), and MUSCLE (version 3.6) (Edgar, “MUSCLE: multiple sequence alignment with high accuracy and high throughput” Nucleic Acids Research 32 (5): 1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

As used herein, the term “variety” means a group of similar plants that by one or more structural features, genetic features, and/or performance can be distinguished from other varieties within the same species. In certain embodiments, the term variety refers to the botanical taxonomic designation whereby variety is ranked below species or subspecies, as well as the legal definition whereby the term “variety” refers to a commercial plant that is protected under the terms outlined in the International Convention for the Protection of New Varieties of Plants.

As used herein, “vector” includes reference to a nucleic acid used in transfection of a host cell and into which can be inserted a polynucleotide. Vectors are often replicons. Expression vectors permit transcription of a nucleic acid inserted therein.

### Polynucleotides and Polypeptides Conferring Fumonisin Detoxification

Fumonisins are mycotoxins produced by Fusarium verticillioides, F. proliferatum, and F. subglutinans. Mycotoxins such as fumonisin can contaminate crops and crop products following fungal colonization of plants by fungi before harvest. To date, 28 fumonisin analogs have been identified with fumonisins B1, B2, and B3 occurring abundantly. Fumonisin B1 (FB1) is a highly toxic fumonisin known to cause diseases such as equine leukoencephalomalacia in horses, hepatocarcinogenesis in rats, and pulmonary edema in swine.

The present disclosure provides polynucleotides and polypeptides isolated from Serratia sp. that confer the ability to detoxify or metabolize fumonisin. In some embodiments, the Serratia sp. comprises Serratia sp. isolate B1072.

In some embodiments, the polypeptide is a synthetic fusion protein comprising one or more enzymes isolated from Serratia sp. capable of metabolizing fumonisin. The fusion enzyme can comprise a glycosyltransferase protein, such as a glycosyltransferase family 4 protein, a lipopolysaccharide biosynthesis protein, a chitinase, and/or a chitin-binding protein. Without being limited by theory, it is believed that the combined activities of the enzymes result in glycosyltransferase and macromolecule catalase activities on fumonisin in two separate reactions. In the first reaction, the glycosyltransferase activity results in the catalysis of the transfer of a glycosyl group (deprotonation) from fumonisin (donor) to lipid (acceptor), resulting in the protonation (hydrolysis) of the lipid molecule (acceptor), leading to the formation of glycolipids. The transfer of a glycosyl group is also thought to result in the formation of glycosidic linkages between the unit molecules of fumonisin. In the second reaction, the high relative molecular mass macromolecule, fumonisin, comprised of repeating units is thought to be broken down to component molecules of lower relative molecular masses. This predicted mechanism of fumonisin metabolism by the fusion gene products described herein is distinct from other enzymes capable of metabolizing fumonisin.

Thus, in embodiments, polynucleotides conferring the ability to detoxify fumonisin comprise polynucleotide constructs encoding a chitin-binding polypeptide, a chitinase polypeptide, a glycosyltransferase polypeptide, and/or a lipopolysaccharide biosynthesis polypeptide. Similarly, in embodiments, synthetic fusion proteins conferring the ability to detoxify fumonisin comprise a chitin-binding protein polypeptide, a chitinase polypeptide, a glycosyltransferase polypeptide, and/or a lipopolysaccharide biosynthesis protein polypeptide. In some embodiments, fusion protein of the present disclosure can comprise chitinase, glycosyltransferase, and lipopolysaccharide biosynthesis polypeptide.

The compositions and methods described herein target the mycotoxin, fumonisin, rather than the pathogenic microbe (Fusarium) itself. Beneficially, this can reduce fungal resistance due to the lack of selection pressure on the fungus. Furthermore, the compositions and methods described herein utilize isolated polynucleotides and polypeptides derived from Serratia sp. rather than using the bacteria itself. This is beneficial because use of the bacteria itself would not be safe or practical.

Polynucleotides and polypeptides conferring the ability to detoxify fumonisin are provided. Such polypeptides include chitin-binding protein polypeptides, such as the one set forth in SEQ ID NO: 2, and variants thereof, chitinase polypeptides, such as the one set forth in SEQ ID NO: 3-6, and variants thereof, glycosyltransferase protein polypeptides, such as the one set forth in SEQ ID NO: 7, and variants thereof, and lipopolysaccharide biosynthesis protein polypeptides, such as the one set forth in SEQ ID NO: 8, and variants thereof. Also provided are polynucleotide sequences encoding fusion proteins comprising such amino acid sequences, including SEQ ID NO: 1, and variants thereof.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode fumonisin detoxification polypeptides described above. Naturally occurring allelic variants can be identified with the use of well-known molecular biology techniques, for example, with polymerase chain reaction (PCR) and hybridization techniques as outlined above. Variant polynucleotides also include synthetically derived polynucleotides, such as those generated, for example, by using site-directed mutagenesis but which still encode polypeptides conferring fumonisin detoxification. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide.

Variants of a particular polynucleotide encoding a fumonisin detoxification polypeptide are encompassed and can be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and algorithms described herein. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

Methods of alignment of sequences for comparison are well known in the art and can be accomplished using mathematical algorithms such as the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; and the algorithm of Karlin and Altschul (1990) Proc. Natl. Acad. Sci. USA 872264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA).

Orthologs” and “paralogs” encompass evolutionary concepts used to describe the ancestral relationships of genes. Paralogs are genes within the same species that have originated through duplication of an ancestral gene; orthologs are genes from different organisms that have originated through speciation, and are also derived from a common ancestral gene.

Those skilled in the art may find further candidate fumonisin resistance genes based on genome synteny and sequence similarity. In one embodiment, additional gene candidates can be obtained by hybridization or PCR using sequences based on the fumonisin resistance nucleotide sequences noted above.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as 32P, or any other detectable marker. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the disclosure as described herein.

With regard to a defined polypeptide, it will be appreciated that % identity figures higher than those provided above will encompass preferred embodiments. Thus, where applicable, in some embodiments, the fumonisin detoxification polypeptide comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and at least 99.9% identical to SEQ ID NO: 2, 3, 4, 5, 6, 7, and/or 8.

By “variant” polypeptide is intended a polypeptide derived from the protein of SEQ ID NO: 2-8, by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

As used herein, a “conservatively modified variant” refers to the substitution of one or more amino acids with a chemically similar amino acid. Conservative substitution tables are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). See also, Creighton (1984) Proteins W.H. Freeman and Company.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Thus, functional variants and fragments of the fumonisin resistance polypeptides, and nucleic acid molecules encoding them, also are within the scope of the present disclosure, and unless specifically described otherwise, irrespective of the origin of the polypeptide and irrespective of whether it occurs naturally.

In addition, one of ordinary skill in the art will further appreciate that changes can be introduced by mutation into the nucleotide sequences thereby leading to changes in the amino acid sequence of the encoded proteins without altering the biological activity of the proteins. Thus, for example, a polynucleotide molecule encoding fumonisin detoxification polypeptides having an amino acid sequence that differs from that of SEQ ID NOs: 2-8 can be created by introducing one or more nucleotide substitutions, additions, or deletions into the corresponding nucleotide sequence, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present disclosure.

A deletion refers to removal of one or more amino acids from a protein. An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag·100 epitope, c-myc epitope, FLAG®-epitope, lacZ, CMP (calmodulin-binding peptide), HA epitope, protein Cepitope VSV epitope, and fluorescent tags such a green fluorescent protein (GFP) or yellow fluorescent protein (YFP).

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues but may be clustered depending upon functional constraints placed upon the polypeptide and may range from 1 to 10 amino acids; insertions will usually be of the order of about 1 to 10 amino acid residues.

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

In certain embodiments, the polypeptides include at least one amino acid substitution, insertion, or deletion so that they do not recite a naturally occurring amino acid sequence.

### Genome Editing

Targeted modification of plant genomes through the use of genome editing methods can be used to modify an endogenous gene through modification of plant genomic DNA. Genome editing methods can also enable targeted insertion of one or more nucleic acids of interest (e.g., a polynucleotide encoding a polypeptide conferring fumonisin resistance) into a plant genome. Methods of genome editing to modify, delete, or insert nucleic acid sequences into genomic DNA are known in the art.

The targeted DNA modification of the genomic locus may be done using any genome modification technique known in the art. In certain embodiments the targeted DNA modification is through a genome modification technique selected from a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpfl endonuclease systems, and the like. In certain embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10:957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

A TALEN comprises a TAL effector DNA binding domain and an endonuclease domain. TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AhvI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or Pl- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure include, for example a Cas9 protein, a Cas 12a protein, a Cas 12b protein, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein the guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9, Cas12a, and Cas12b endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crRNA sequence linked to a tracrRNA sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crRNA and the tracrRNA may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein the guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In certain embodiments, the variable targeting domain comprises a contiguous stretch of 12 to 30 nucleotides. The variable targeting domain can be composed of a DNA sequence, an RNA sequence, a modified DNA sequence, a modified RNA sequence, or any combination thereof.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein the guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein the guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in the cell. The specific promoter can be, but is not limited to, a RNA polymerase III promoter, which allow for transcription of RNA with precisely defined, unmodified, 5′- and 3′-ends (DiCarlo et al., Nucleic Acids Res. 41:4336-4343; Ma et al., Mol. Ther. Nucleic Acids 3: e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to direct a guide polynucleotide/Cas endonuclease complex to a unique DNA target site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

Various methods and compositions can be employed to obtain a cell or organism having a polynucleotide of interest inserted in a target site. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

The region of homology on the donor DNA can have homology to any sequence flanking the target site. While in certain embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In certain embodiments, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest, insertion of polynucleotides of interest, gene knock-out, gene-knock in, modification of splicing sites and/or introducing alternate splicing sites, and modifications of nucleotide sequences encoding a protein of interest.

### Expression Constructs

Polynucleotides as described herein can be provided in an expression construct. Expression constructs generally include regulatory elements that are functional in the intended host cell in which the expression construct is to be expressed. Thus, a person of ordinary skill in the art can select regulatory elements for use in bacterial host cells, yeast host cells, plant host cells, insect host cells, and mammalian host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, “operably linked” means two DNA molecules linked in manner so that one may affect the function of the other. Operably-linked DNA molecules may be part of a single contiguous molecule and may or may not be adjacent. For example, a promoter is operably linked with a polypeptide-encoding DNA molecule in a DNA construct where the two DNA molecules are so arranged that the promoter may affect the expression of the DNA molecule.

As used herein, the term “heterologous” refers to the relationship between two or more items derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell, seed, or organism into which it is inserted when it would not naturally occur in that particular cell, seed, or organism.

An expression construct can comprise a promoter sequence operably linked to a polynucleotide sequence encoding a fumonisin detoxification polypeptide as described herein. Promoters can be incorporated into a polynucleotide using standard techniques known in the art. Multiple copies of promoters or multiple promoters can be used in an expression construct as described herein. In certain embodiments, a promoter can be positioned about the same distance from the transcription start site in the expression construct as it is from the transcription start site in its natural genetic environment. Some variation in this distance is permitted without a substantial decrease in promoter activity. A transcription start site is typically included in the expression construct.

Embodiments relate to a polynucleotide encoding a fumonisin detoxification polypeptide, wherein the polynucleotide is further defined as operably linked to a heterologous regulatory element. In certain embodiments, the heterologous regulatory element is a promoter functional in a plant cell.

If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, zein promoters including maize zein promoters, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-la promoter, ubiquitin promoter, actin promoter, alcA gene promoter, pin2 promoter (Xu et al., 1993), maize Wipl promoter, maize trpA gene promoter (U.S. Pat. No. 5,625,136), maize CDPK gene promoter, and RUBISCO SSU promoter (U.S. Pat. No. 5,034,322) can also be used. Constitutive promoters (such as the CaMV, ubiquitin, actin, or NOS promoter), developmentally regulated promoters, and inducible promoters (such as those promoters than can be induced by heat, light, hormones, or chemicals) are also contemplated for use with polynucleotide expression constructs described herein.

Expression constructs may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides described herein. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

Optionally the polynucleotide encoding the fumonisin detoxification polypeptide is codon-optimized to remove features inimical to expression and codon usage is optimized for expression in the particular crop (see, for example, U.S. Pat. No. 6,051,760; EP 0359472; EP 80385962; EP 0431829; and Perlak et al. (1991) PNAS USA 88:3324-3328; all of which are herein incorporated by reference).

In certain embodiments, the polynucleotides include at least one nucleotide substitution, insertion, or deletion so that they do not recite a naturally occurring nucleic acid sequence.

### Transformation Methods

Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, which is incorporated herein by reference in its entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or an herbicide. Any of the herbicides to which plants of this disclosure can be resistant is an agent for selective markers. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant Varieties with the Ability to Detoxify Fumonisin

Several embodiments relate to plant varieties having the ability to detoxify fumonisin. Plants, plant parts, and seeds of these varieties can comprise a heterologous polynucleotide encoding polypeptides of the present disclosure, wherein expression of the polynucleotide confers the ability to detoxify fumonisin. Plants may be monocots or dicots, and may include, for example, maize, rice, wheat, barley, rye, sorghum, soybean, oat, millet plant, or any plant prone to fumonisin contamination.

Plants that are particularly useful in the methods of the present disclosure include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. In certain embodiments, the plant is a crop plant. Examples of crop plants include inter alia maize, rice, wheat, barley, rye, sorghum, soybean, oat, or millet plant.

Also provided are a progeny or a descendant of a fumonisin resistant plant as well as seeds derived from the fumonisin resistant plants and cells derived from the fumonisin resistant plants as described herein.

The present disclosure also provides a progeny or descendant plant derived from a plant comprising in at least some of its cells a polynucleotide encoding a polypeptide, wherein expression of the polynucleotide confers to the progeny or descendant plant the ability to detoxify fumonisin.

In certain embodiments, seeds of the present disclosure comprise the fumonisin detoxification characteristics of the fumonisin resistant plant. In certain embodiments, a seed is capable of germination into a plant comprising in at least some of its cells a polynucleotide encoding a polypeptide, wherein expression of the polynucleotide confers to the progeny or descendant plant the ability to detoxify fumonisin.

In certain embodiments, plant cells of the present disclosure are capable of regenerating a plant or plant part. In certain embodiments, plant cells are not capable of regenerating a plant or plant part. Examples of cells not capable of regenerating a plant include, but are not limited to, endosperm, seed coat (testa and pericarp), and root cap.

Several embodiments provide a commodity plant product prepared from the fumonisin resistant plants. Examples of commodity plant products include, without limitation, grain, oil, and meal. In one embodiment, a plant product is plant grain (e.g., grain suitable for use as feed or for processing), plant oil (e.g., oil suitable for use as food or biodiesel), or plant meal (e.g., meal suitable for use as feed). For example, the commodity plant product can comprise fodder, seed meal, oil, milk (e.g., soy milk), flour (e.g., soy flour), grits, protein (e.g., protein concentrate, hydrolyzed vegetable protein, textured vegetable protein), tofu, miso, tempeh, fiber, starch, bio-composite building materials (e.g., particleboard, laminated plywood, or lumber products), or seed-treatment-coated seeds. The commodity plant products can comprise the fumonisin detoxification protein or the polynucleotide encoding the fumonisin detoxification protein.

The commodity plant product may be produced at the site where the plant has been grown, the plants and/or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the method is performed, while allowing repeated times the steps of product production e.g., by repeated removal of harvestable parts of the plants of the disclosure and if necessary further processing of these parts to arrive at the product. It is also possible that the step of growing the plants is repeated and plants or harvestable parts are stored until the production of the product is then performed once for the accumulated plants or plant parts. Also, the steps of growing the plants and producing the product may be performed with an overlap in time, even simultaneously to a large extent or sequentially. Generally, the plants are grown for some time before the product is produced.

### Gene Stacking

The fumonisin detoxification genes of the disclosure can be stacked with any combination of polynucleotide sequences of interest in order to create plants with a desired phenotype. For example, the fumonisin detoxification genes may be stacked with one or more additional fungal resistance genes or an herbicide tolerance gene.

By way of example, polynucleotides that may be stacked with the fumonisin detoxification genes include nucleic acids encoding polypeptides conferring resistance to pests/pathogens such as viruses, nematodes, insects or fungi, and the like. Illustrative polynucleotides that may be stacked with the fumonisin genes of the disclosure include polynucleotides encoding: polypeptides having pesticidal and/or insecticidal activity, such as Bacillus thuringiensis toxic proteins (described in U.S. Pat. Nos. 5,366,892; 5,747,450; 5,737,514; 5,723,756; 5,593,881; and Geiser et al., (1986) Gene 48:109), lectins (Van Damme et al. (1994) Plant Mol. Biol. 24:825, pentin (described in U.S. Pat. No. 5,981,722), and the like; traits desirable for disease or herbicide resistance (e.g., additional fumonisin detoxification genes (U.S. Pat. No. 5,792,931); avirulence and disease resistance genes (Jones et al. (1994) Science 266:789; Martin et al., (1993) Science 262:1432; Mindrinos et al. (1994) Cell 78:1089); acetolactate synthase (ALS) mutants that lead to herbicide resistance such as the S4 and/or Hra mutations; glyphosate resistance (e.g., 5-enol-pyrovyl-shikimate-3-phosphate-synthase (EPSPS) gene, described in U.S. Pat. Nos. 4,940,935 and 5,188,642; or the glyphosate N-acetyltransferase (GAT) gene, described in Castle et al. (2004) Science, 304:1151-1154; and in U.S. Patent App. Pub. Nos. 20070004912, 20050246798, and 20050060767)); glufosinate resistance (e.g, phosphinothricin acetyl transferase genes PAT and BAR, described in U.S. Pat. Nos. 5,561,236 and 5,276,268); resistance to herbicides including sulfonyl urea, DHT (2,4D), and PPO herbicides (e.g., glyphosate acetyl transferase, aryloxy alkanoate dioxygenase, acetolactate synthase, and protoporphyrinogen oxidase); other cytochrome P450s that confer herbicide tolerance (U.S. patent application Ser. No. 12/156,247; U.S. Pat. Nos. 6,380,465; 6,121,512; 5,349,127; 6,649,814; and 6,300,544; and PCT Patent App. Pub. No. WO2007000077); and traits desirable for processing or process products such as high oil (e.g., U.S. Pat. No. 6,232,529); modified oils (e.g., fatty acid desaturase genes (U.S. Pat. No. 5,952,544; WO 94/11516)); and modified starches (e.g., ADPG pyrophosphorylases (AGPase), starch synthases (SS), starch branching enzymes (SBE), and starch debranching enzymes (SDBE)); the disclosures of which are herein incorporated by reference.

In certain embodiments, the plant comprises at least one herbicide-tolerant trait selected, for example, from 5-enolpyruvylshikimate-3-phosphate synthase (EPSPS), Glyphosate acetyl transferase (GAT), phosphinothricin acetyltransferase (PAT), Acetohydroxyacid synthase (AHAS; EC 4.1.3.18, also known as acetolactate synthase or ALS), hydroxyphenyl pyruvate dioxygenase (HPPD), Phytoene desaturase (PD) and dicamba degrading enzymes as disclosed in WO 02/068607, or phenoxyacetic acid- and phenoxypropionic acid-derivative degrading enzymes as disclosed in WO 2008141154 or WO 2005107437.

### Use in Breeding Methods

The plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity and plant and ear height is desirable. Traditional plant breeding is an important tool in developing new and improved commercial crops. This disclosure encompasses methods for producing a plant by crossing a first parent plant with a second parent plant wherein one or both of the parent plants is a plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of hybrids in a plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed plant to an elite inbred line and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

### Fumonisin Contamination Control

Several embodiments provide compositions and methods for controlling fumonisin contamination at a plant cultivation site by contacting a plant at the site with a composition that comprises a polynucleotide encoding a fumonisin detoxification polypeptide or fusion protein. Other embodiments comprise providing at the site a plant comprises a polynucleotide encoding a fumonisin detoxification polypeptide or fusion protein.

In certain embodiments, the fumonisin detoxification polypeptide is isolated from Serratia sp. In some embodiments, the Serratia sp. is Serratia sp. isolate B1072. In some embodiments, the fumonisin detoxification polypeptide comprises a chitin-binding protein, a chitinase, a glycosyltransferase protein, and/or a lipopolysaccharide biosynthesis protein. In some embodiments, the polynucleotide encoding the polypeptide has at least 70%, at least 80%, at least 90%, at least 95%, at least 98%, at least 99%, or 100% sequence identity to SEQ ID NO: 1. In some embodiments, the polynucleotide encodes a polypeptide having at least 70%, at least 80%, at least 90%, at least 95%, at least 98%, at least 99%, or 100% sequence identity to SEQ ID NO: 2, 3, 4, 5, 6, 7, and/or 8.

In certain embodiments, the compositions and methods can comprise permeability-enhancing agents and treatments to condition the surface of plant tissue, e.g., leaves, stems, roots, flowers, or fruits, to permeation by the polynucleotides into plant cells. The transfer of polynucleotides into plant cells can be facilitated by the prior or contemporaneous application of a polynucleotide to the plant tissue. In some embodiments the permeability-enhancing agent is applied subsequent to the application of the polynucleotide composition. The permeability-enhancing agent enables a pathway for polynucleotides through cuticle wax barriers, stomata and/or cell wall or membrane barriers and into plant cells. Suitable agents to facilitate transfer of the composition into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof.

Chemical agents for conditioning include (a) surfactants, (b) an organic solvent or an aqueous solution or aqueous mixtures of organic solvents, (c) oxidizing agents, (c) acids, (f) bases, (g) oils, (h) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Such agents for conditioning of a plant to permeation are applied to the plant by any convenient method, e.g., spraying or coating with a powder, emulsion, suspension, or solution; similarly, the polynucleotide molecules are applied to the plant by any convenient method, e.g., spraying or wiping a solution, emulsion, or suspension.

### Detection Tools

Several embodiments provide a method for identifying a plant having the ability to detoxify fumonisin, or cells or tissues thereof. In certain embodiments, the method includes using primers or probes which specifically recognize a portion of the sequence of the fumonisin detoxification gene of the disclosure. In certain embodiments, the identification is performed using polymerase chain reaction. Several embodiments provide kits for identifying a plant having the ability to detoxify fumonisin.

Probes and primers are provided which are of sufficient nucleotide length to bind specifically to the target DNA sequence under the reaction or hybridization conditions. Suitable probes and primers are at least 10, 11, 12, 13, 14, 15, 16, 17, 18, 19, 20, 21, 22, 23, 24, 25, 26, 27, 28, 29 or 30 nucleotides in length, and less than 35, 34, 33, 32, 31, 30, 29, 28, 27, 26, 2, 5 2, 4 23, 22, 21, 20, 19, 18, 17, 16, 15, 14, 13, or 12 nucleotides in length. Such probes and primers can hybridize specifically to a target sequence under high stringency hybridization conditions. In certain embodiments, probes and primers have complete or 100% DNA sequence similarity of contiguous nucleotides with the target sequence, although probes which differ from the target DNA sequence but retain the ability to hybridize to target DNA sequence may also be used. Reverse complements of the primers and probes disclosed herein are also provided and can be used in the methods and compositions described herein.

The methods, kits, and primers can be used for different purposes including, but not limited to the following: identifying the presence or absence of the ability to detoxify fumonisin in plants, plant material such as seeds or cuttings; and tailoring a fungicidal regime to effectively and economically manage fungal contamination affecting agricultural crops.

### Deposit

A deposit of the Serratia sp. isolate B1072 is and has been maintained by The Texas A&M University System, 3369 TAMU, College Station, TX 77843, since prior to the filing date of this application. Access to this deposit will be available during the pendency of the application to the Commissioner of Patents and Trademarks and persons determined thereby to be entitled thereto upon request. Deposit will be made in a timely manner upon allowance of any claims in the application, whereby, the Applicant(s) will make available to the public, pursuant to 37 CFR 1.808 (2), a deposit of at 11 vials of Serratia sp. isolate B1072 with the Provasoli-Guillard National Center for Marine Algae and Microbiota (NCMA), 60 Bigelow Drive, East Boothbay, Me. 04544, USA, with NCMA Accession No. The seeds deposited with the NCMA on ______ will be taken from the same deposit maintained at The Texas A&M University System and described above. Additionally, Applicant(s) will meet all the requirements of 37 C.F.R. § 1.801-1.809, including providing an indication of the viability of the sample when the deposit is made. These deposits will be maintained in the NCMA depository, which is a public depository, for a period of 30 years, or 5 years after the most recent request, or for the enforceable life of the patent, whichever is longer, and will be replaced if it ever becomes nonviable during that period. Applicant has no authority to waive any restrictions imposed by law on the transfer of biological material or its transportation in commerce. Applicant does not waive any infringement of its rights granted under this patent or under the Plant Variety Protection Act (7 USC 2321 et seq.).

### Embodiments

The following numbered embodiments also form part of the present disclosure:

1. A polynucleotide construct that confers fumonisin detoxification in plants, encoding a chitin-binding polypeptide, a chitinase polypeptide, a glycosyltransferase polypeptide, and a lipopolysaccharide biosynthesis polypeptide, wherein the polynucleotide has at least 70%, at least 80%, at least 90%, at least 95%, at least 98%, at least 99%, or 100% sequence identity to SEQ ID NO: 1.

2. The polynucleotide construct of embodiment 1, wherein the chitin-binding polypeptide comprises an amino acid sequence having at least 70%, at least 80%, at least 90%, at least 95%, at least 98%, at least 99%, or 100% sequence identity to SEQ ID NO: 2.

3. The polynucleotide construct of embodiment 1 or 2, wherein the chitinase polypeptide comprises an amino acid sequence having at least 70%, at least 80%, at least 90%, at least 95%, at least 98%, at least 99%, or 100% sequence identity to SEQ ID NO: 3, 4, 5, and/or 6.

4. The polynucleotide construct of any one of embodiments 1-3, wherein the glycosyltransferase polypeptide comprises an amino acid sequence having at least 70%, at least 80%, at least 90%, at least 95%, at least 98%, at least 99%, or 100% sequence identity to SEQ ID NO: 7.

5. The polynucleotide construct of any one of embodiments 1-4, wherein the lipopolysaccharide biosynthesis polypeptide comprises an amino acid sequence having at least 70%, at least 80%, at least 90%, at least 95%, at least 98%, at least 99%, or 100% sequence identity to SEQ ID NO: 8.

6. A polynucleotide construct that confers fumonisin detoxification in plants, wherein the polynucleotide construct encodes: a chitin-binding polypeptide, wherein the chitin-binding polypeptide comprises an amino acid sequence having at least 70%, at least 80%, at least 90%, at least 95%, at least 98%, at least 99%, or 100% sequence identity to SEQ ID NO: 2; a chitinase polypeptide, wherein the chitinase polypeptide comprises an amino acid sequence having at least 70%, at least 80%, at least 90%, at least 95%, at least 98%, at least 99%, or 100% sequence identity to SEQ ID NO: 3, 4, 5, and/or 6; a glycosyltransferase polypeptide, wherein the glycosyltransferase polypeptide comprises an amino acid sequence having at least 70%, at least 80%, at least 90%, at least 95%, at least 98%, at least 99%, or 100% sequence identity to SEQ ID NO: 7; and a lipopolysaccharide biosynthesis polypeptide, wherein the lipopolysaccharide biosynthesis polypeptide comprises an amino acid sequence having at least 70%, at least 80%, at least 90%, at least 95%, at least 98%, at least 99%, or 100% sequence identity to SEQ ID NO: 8.

7. The polynucleotide construct of embodiment 6, wherein the polynucleotide construct has at least 70%, at least 80%, at least 90%, at least 95%, at least 98%, at least 99%, or 100% sequence identity to SEQ ID NO: 1.

8. The polynucleotide construct of any one of embodiments 1-7, wherein the polynucleotide is operably linked to a heterologous promoter functional in a plant cell.

9. The polynucleotide construct of any one of embodiments 1-8, wherein the polynucleotide construct encodes a fusion protein comprising the chitin-binding polypeptide, chitinase polypeptide, glycosyltransferase polypeptide, and/or lipopolysaccharide biosynthesis polypeptide.

10. A vector comprising the polynucleotide construct of any one of embodiments 1-9.

11. A biological sample comprising the polynucleotide construct of any one of embodiments 1-9.

12. A cell comprising the polynucleotide construct of any one of embodiments 1-9.

13. The cell of embodiment 12, wherein the cell is a plant cell.

14. The cell of embodiment 12, wherein the cell is a bacterial cell.

15. The cell of embodiment 14, wherein the cell is not a Serratia sp. cell.

16. A plant, plant part, plant seed, or plant cell comprising the polynucleotide construct of any one of embodiments 1-9.

17. An antifungal composition comprising the polynucleotide construct of any one of embodiments 1-9.

18. A method for controlling fumonisin contamination at a plant cultivation site, the method comprising: contacting a plant at the plant cultivation site with an antifungal composition of embodiment 17.

19. The method of embodiment 18, wherein the composition further comprises a permeability-enhancing agent.

20. A plant variety having an ability to detoxify fumonisin, wherein a plant, a plant part, or a seed of said variety comprises a heterologous polynucleotide construct encoding a chitin-binding polypeptide, a chitinase polypeptide, a glycosyltransferase polypeptide, and a lipopolysaccharide biosynthesis polypeptide, wherein the chitin-binding polypeptide, chitinase polypeptide, glycosyltransferase polypeptide, and lipopolysaccharide biosynthesis polypeptide are Serratia sp. polypeptides.

21. The plant variety of embodiment 20, wherein the Serratia sp. comprises Serratia sp. isolate B1072.

22. The plant variety of embodiment 20 or 21, wherein the heterologous polynucleotide has at least 70%, at least 80%, at least 90%, at least 95%, at least 98%, at least 99%, or 100% sequence identity to SEQ ID NO: 1.

23. The plant variety of any one of embodiments 20-22, wherein the chitin-binding polypeptide comprises an amino acid sequence having at least 70%, at least 80%, at least 90%, at least 95%, at least 98%, at least 99%, or 100% sequence identity to SEQ ID NO: 2.

24. The plant variety of any one of embodiments 20-24, wherein the chitinase polypeptide comprises an amino acid sequence having at least 70%, at least 80%, at least 90%, at least 95%, at least 98%, at least 99%, or 100% sequence identity to SEQ ID NO: 3, 4, 5, and/or 6.

25. The plant variety of any one of embodiments 20-24, wherein the glycosyltransferase polypeptide comprises an amino acid sequence having at least 70%, at least 80%, at least 90%, at least 95%, at least 98%, at least 99%, or 100% sequence identity to SEQ ID NO: 7.

26. The plant variety of any one of embodiments 20-25, wherein the lipopolysaccharide biosynthesis polypeptide comprises an amino acid sequence having at least 70%, at least 80%, at least 90%, at least 95%, at least 98%, at least 99%, or 100% sequence identity to SEQ ID NO: 8.

27. The plant variety of any one of embodiments 21-26, wherein the polynucleotide is operably linked to a heterologous promoter functional in a plant cell.

28. The plant variety of any one of embodiments 21-27, wherein the fumonisin is fumonisin B1.

29. The plant variety of any one of embodiments 21-28, wherein the plant is a dicotyledonous or monocotyledonous plant.

30. The plant variety of any one of embodiments 21-29, wherein the plant is a maize, rice, wheat, barley, rye, sorghum, soybean, oat, or millet plant.

31. The plant variety of any one of embodiments 21-30, wherein the plant further comprises a second fungal-resistant trait.

32. The plant variety of any one of embodiments 21-31, wherein the plant variety comprises elite germplasm.

33. A plant, a plant part, a seed, or a plant cell of the plant variety of any one of embodiments 21-32.

34. A method for controlling fumonisin contamination at a plant cultivation site, the method comprising: providing at the site a plant of embodiment 33.

35. A commodity plant product prepared from the plant, plant part, plant seed, or plant cell of embodiment 33.

36. The commodity plant product of embodiment 35, wherein the product comprises fodder, seed meal, oil, milk, flour, grits, protein, fiber, tofu, tempeh, miso, starch, bio-composite building materials or seed-treatment-coated seed.

37. A method for producing a commodity plant product, the method comprising processing the plant, plant part, plant seed, or plant cell of embodiment 33 to obtain the product.

38. A method for producing a plant, plant part, or plant cell with an ability to detoxify fumonisin, the method comprising: modifying the plant to comprise a heterologous polynucleotide construct encoding a chitin-binding polypeptide, a chitinase polypeptide, a glycosyltransferase polypeptide, and a lipopolysaccharide biosynthesis polypeptide, wherein the chitin-binding protein polypeptide, chitinase polypeptide, glycosyltransferase protein polypeptide, and lipopolysaccharide biosynthesis protein polypeptide are Serratia sp. polypeptides.

39. The method of embodiment 38, wherein the method comprises introducing a genome editing system into the plant, plant part, or plant cell.

40. The method of embodiment 38 or 39, wherein the genome editing system comprises a CRISPR/Cas system, a TALEN, or a zinc finger nuclease.

41. The method of any one of embodiments 38-40, wherein the Serratia sp. comprises Serratia sp. isolate B1072.

42. The method of any one of embodiments 38-41, wherein the chitin-binding polypeptide comprises an amino acid sequence having at least 70%, at least 80%, at least 90%, at least 95%, at least 98%, at least 99%, or 100% sequence identity to SEQ ID NO: 2.

43. The method of any one of embodiments 38-42, wherein the chitinase polypeptide comprises an amino acid sequence having at least 70%, at least 80%, at least 90%, at least 95%, at least 98%, at least 99%, or 100% sequence identity to SEQ ID NO: 3, 4, 5, and/or 6.

44. The method of any one of embodiments 38-43, wherein the glycosyltransferase polypeptide comprises an amino acid sequence having at least 70%, at least 80%, at least 90%, at least 95%, at least 98%, at least 99%, or 100% sequence identity to SEQ ID NO: 7.

45. The method of any one of embodiments 38-44, wherein the lipopolysaccharide biosynthesis polypeptide comprises an amino acid sequence having at least 70%, at least 80%, at least 90%, at least 95%, at least 98%, at least 99%, or 100% sequence identity to SEQ ID NO: 8.

46. The method of any one of embodiments 38-45, wherein the heterologous polynucleotide construct has at least 70%, at least 80%, at least 90%, at least 95%, at least 98%, at least 99%, or 100% sequence identity to SEQ ID NO: 1.

47. The method of any one of embodiments 38-46, wherein the fumonisin is fumonisin B1.

48. The method of any one of embodiments 38-47, wherein the plant is a dicotyledonous or monocotyledonous plant.

49. The method of any one of embodiments 38-48, wherein the plant is a maize, rice, wheat, barley, rye, sorghum, soybean, oat, or millet plant.

50. A method for identifying a plant having an ability to detoxify fumonisin, the method comprising: obtaining a nucleic acid sample from a plant suspected of having the ability to detoxify fumonisin; detecting in the sample a polynucleotide construct that confers fumonisin detoxification in plants encoding a chitin-binding polypeptide, a chitinase polypeptide, a glycosyltransferase polypeptide, and a lipopolysaccharide biosynthesis polypeptide; and determining that the plant has an increased resistance to fumonisin based on the presence of the polynucleotide.

51. The method of embodiment 50, wherein the chitin-binding polypeptide, chitinase polypeptide, glycosyltransferase polypeptide, and lipopolysaccharide biosynthesis polypeptide are Serratia sp. proteins.

52. The method of embodiment 50 or 51, wherein the Serratia sp. comprises Serratia sp. isolate B1072.

53. The method of any one of embodiments 50-52, wherein the polynucleotide has at least 70%, at least 80%, at least 90%, at least 95%, at least 98%, at least 99%, or 100% sequence identity to SEQ ID NO: 1.

54. The method of any one of embodiments 50-53, wherein the chitin-binding polypeptide comprises an amino acid sequence having at least 70%, at least 80%, at least 90%, at least 95%, at least 98%, at least 99%, or 100% sequence identity to SEQ ID NO: 2.

55. The method of any one of embodiments 50-54, wherein the chitinase polypeptide comprises an amino acid sequence having at least 70%, at least 80%, at least 90%, at least 95%, at least 98%, at least 99%, or 100% sequence identity to SEQ ID NO: 3, 4, 5, and/or 6.

56. The method of any one of embodiments 50-55, wherein the glycosyltransferase polypeptide comprises an amino acid sequence having at least 70%, at least 80%, at least 90%, at least 95%, at least 98%, at least 99%, or 100% sequence identity to SEQ ID NO: 7.

57. The method of any one of embodiments 50-56, wherein the lipopolysaccharide biosynthesis polypeptide comprises an amino acid sequence having at least 70%, at least 80%, at least 90%, at least 95%, at least 98%, at least 99%, or 100% sequence identity to SEQ ID NO: 8.

58. The method of any one of embodiments 50-57, wherein the detecting comprises amplifying the polynucleotide using at least two primers.

59. The method of any one of embodiments 50-58, wherein the plant is a maize, rice, wheat, barley, rye, sorghum, soybean, oat, or millet plant.

60. A kit for identifying a plant having an ability to detoxify fumonisin, the kit comprising at least two primers, wherein the at least two primers recognize a polynucleotide encoding a chitin-binding polypeptide, a chitinase polypeptide, a glycosyltransferase polypeptide, and a lipopolysaccharide biosynthesis polypeptide, wherein the chitin-binding polypeptide, chitinase polypeptide, glycosyltransferase polypeptide, and lipopolysaccharide biosynthesis polypeptide are Serratia sp. proteins.

### EXAMPLES

### Example 1

A survey was conducted of the major corn production areas in three states including the Panhandle and South Plains of Texas, the Northwest and Southwest areas of Kansas, and the East Central and Northeast areas of Colorado respectively, for bacteria with potential to inhibit fumonisin production by fumonisin-producing fusarium pathogens on corn. Corn samples were collected in Texas, Kansas, and Colorado once corn plants in the respective states reached the VT (tasseling) growth stage. A wide variance of fumonisin levels were seen. In the Panhandle for instance, the minimum/maximum in parts per million (ppm) levels recorded included Hartley (60/60 ppm), Dallam (0.6/64.8 ppm), Sherman (5/81 ppm), Potter (4.2/214.4 ppm) and Randall (8.6/165.5 ppm). In the South Plains, similar variabilities were detected, for instance, Parmer (3/106 ppm), Hale (30/104 ppm), Castro (4/40 ppm), Lamb (0.2/72 ppm), and Swisher (106/106 ppm). Although the reason for the wide variability within and across the counties has not been established, potential factors include differences in corn varieties planted and their interactions with their respective local environments, differences in agronomic practices, and even differences in plant endophytes across locations. Indeed, some bacterial endophytes are known to confer such functional benefits as promotion of root growth, nitrogen fixation, phosphate solubilization, and control of pathogens and associated toxins to their host plants.

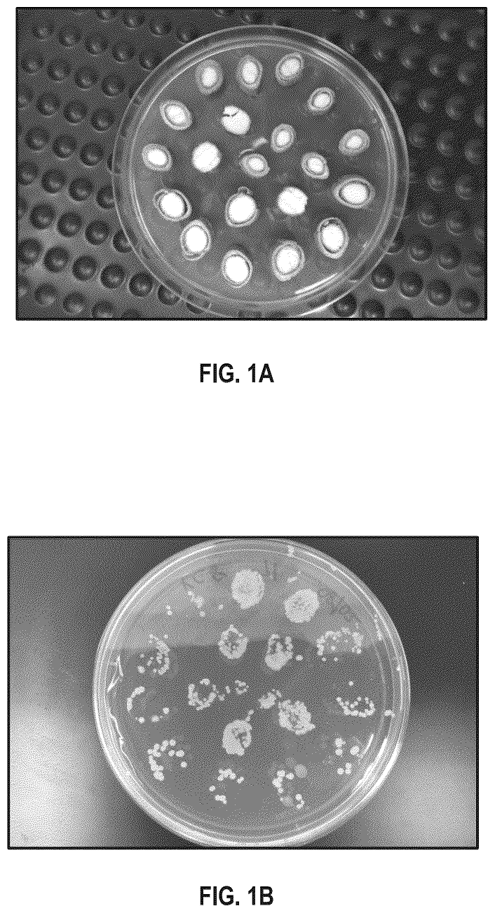

Corn plants collected from each state were processed to isolate resident bacteria population from individual corn stalks (FIGS. 1A-B). The isolated bacteria were subsequently stored at the ultra-low temperature of −80° C. pending further analysis and evaluations.

A total of 6,432 bacterial consortia were isolated and stored in −80° C. Each consortium consisted of on average three distinct types of bacteria, to give a total of 19,296 bacteria colonies. All 6,432 bacterial consortia were evaluated for potential antifungal properties. Specifically, they were evaluated for their potential to inhibit growth of mycotoxigenic Fusarium.

242 consortia demonstrated reproducible inhibition of fungal growth in-vitro. A total of 50 consortia representing a subset of the 242 consortia were further evaluated and a total of 13 consortia were identified with anti-fumonisin phenotype.

### Example 2

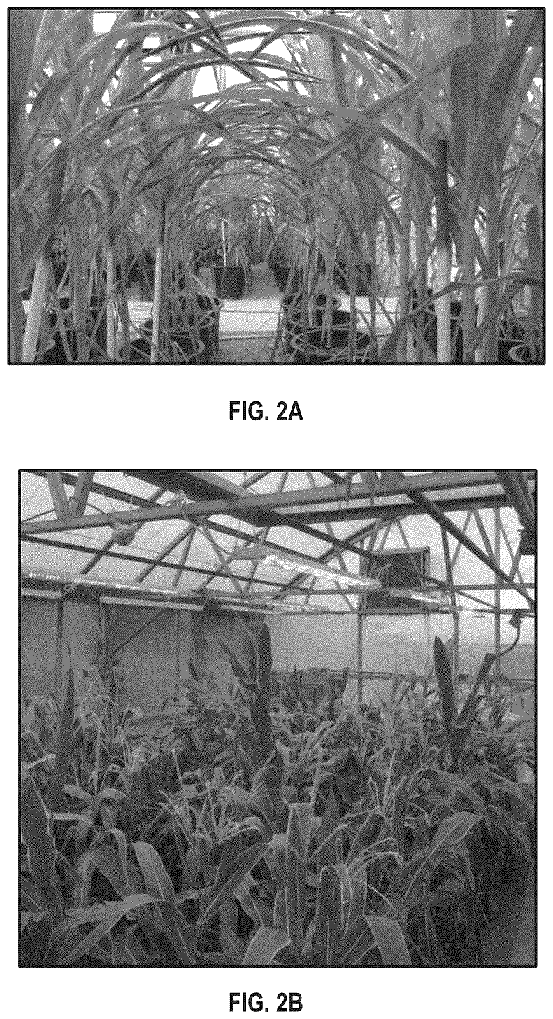

Pioneer P1690AM corn hybrid was planted in Berger BM1 Nutrient Retention General Purpose Media in pots and maintained under greenhouse conditions (FIGS. 2A-B) in two independent studies in Bushland, TX. In the first study, twelve bacteria consortia, selected for evaluation based on results of in-vitro studies, conducted under laboratory conditions in Amarillo, in which they showed potential to mitigate fumonisin accumulation following co-culture with a fumonisin producing Fusarium proliferatum isolate KC8-3 from corn. In the second study, six bacterial consortia, mostly of Texas origin, were selected and evaluated in an expanded study. In both studies, the selected bacterial consortia were evaluated for their potential to mitigate fumonisin accumulation on corn ears inoculated with the F. proliferatum KC8-3 isolate in the greenhouse.

### Experimental Design

### Study 1

In the first study, 12 bacterial consortia (Table 1) were separately used to inoculate corn ears at the VT (tassel) growth stage, in mixtures with conidial suspension of the toxigenic Fusarium isolate KC8-3. Each corn treatment consisted of two corn plants plant per pots with two replicate pots per treatment (FIG. 2A). The treatment pots arrangements were completely randomized in the greenhouse.

TABLE 1
List of Bacterial Consortia

| Bacterial | Source |
|---|---|
| consortia | location |
| 4 | Texas |
| 9 | Texas |
| 10 | Texas |
| 12 | Texas |
| 18 | Kansas |
| 19 | Kansas |
| 25 | Texas |
| 26 | Texas |
| 29 | Texas |
| 33 | Kansas |
| 43 | Colorado |
| 44 | Colorado |

All plants in this study were maintained under natural daylight and night cycles and between 78-80° F. At maturity, corn ears were processed for fumonisin extraction and quantification. Briefly, dried corn ears were shelled, and the kernels milled with a coffee grinder using the Turkish setting. Next, each milled sample was sieved with a #20 sieve mesh and the resultant sample subsequently homogenized using a food mixer for 15 minutes. Subsamples were weighed out from each mixed sample and process for fumonisin detection and quantification using the Envirologix mycotoxin detection and quantification kit and platform. The detection range of the system used is 0.1-100 ppm (parts per million) of total fumonisin. Samples were quantified in duplicates and the resulting values for fumonisin levels analyzed statistically for comparison of treatment effects on fumonisin accumulation in the treated corn ears.

### Study 2

In the second study, the experimental setup in the greenhouse was similar to that from the first study. Treatments consisted of two replicate pots each, arranged in a completely randomized design. Unlike in the first study however, supplemental LED lighting (FIG. 2B) was used during the second study due to decreased daylength period of the winter months when the study was conducted. Plants were similarly maintained at 78-80° F. and a 16-hr photoperiod. In order to allow for an expanded evaluation of the potential of the bacterial consortia, a total of six bacterial consortia, comprising mainly of Texas-sourced consortia as well as two Kansas consortia were selected for evaluation in this study. Consequently, in addition to treatments comprising, separately, of mixtures of each bacteria consortia with conidial suspension of the toxigenic Fusarium isolate KC8-3, two additional treatment types were included in this study. Specifically, inoculations of the bacterial consortia followed by (after 96 hours) inoculation of the fungus as well as inoculations of the bacterial consortia 96 hours after inoculations with the fungus were evaluated.

### Results

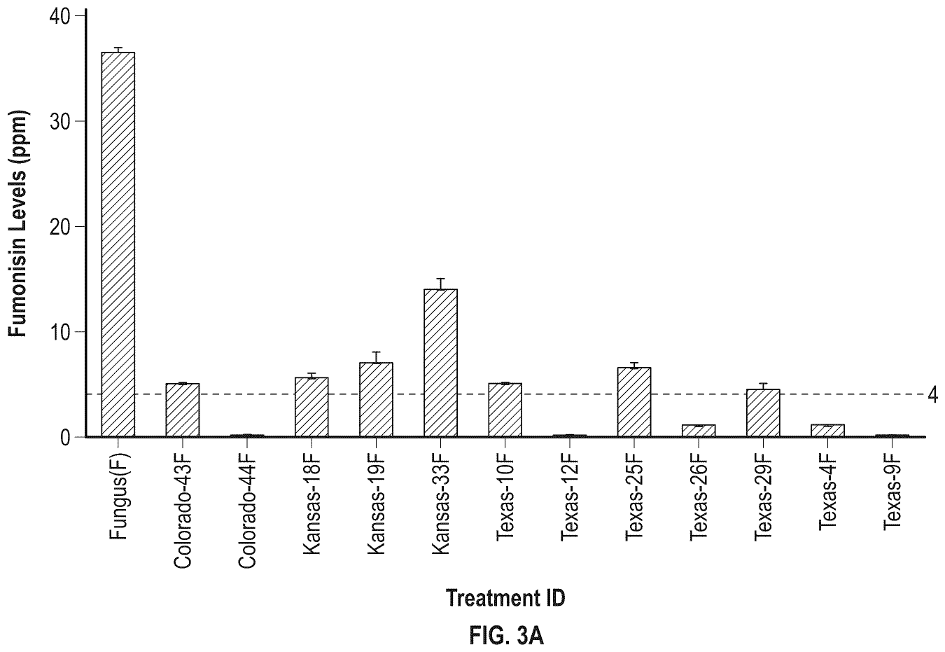

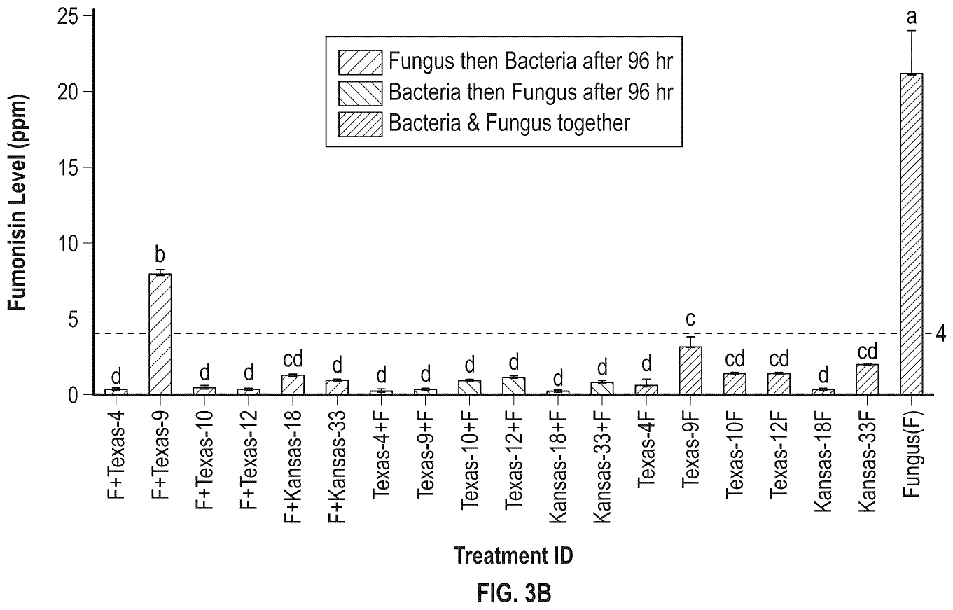

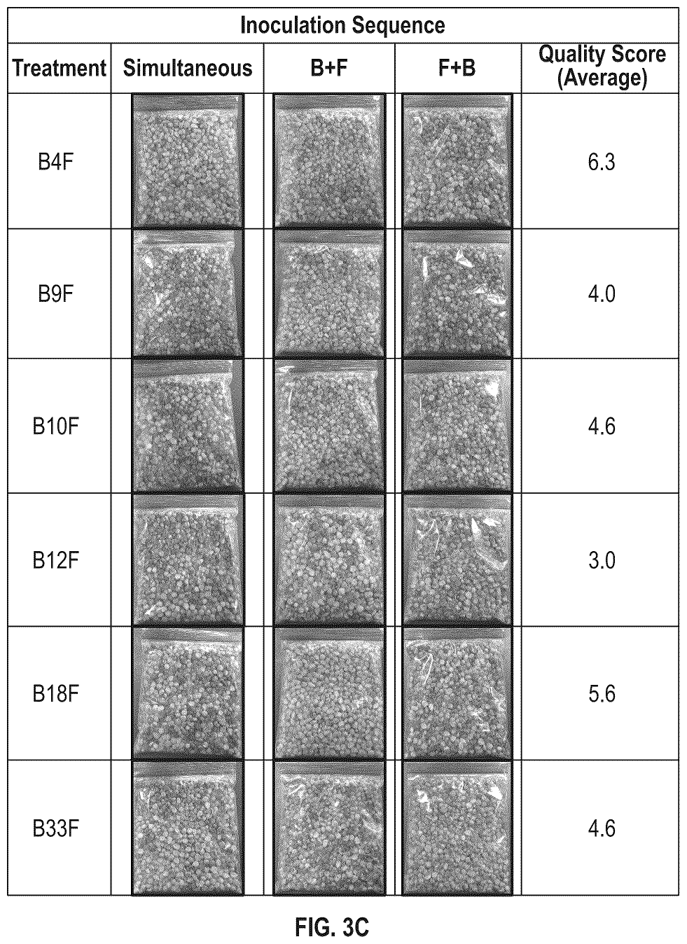

The results of total fumonisin levels quantified in the treatments in the first study showed that of the twelve bacterial consortia, only five resulted in an averaged total fumonisin levels below 4 ppm (FIG. 3A). Four of the five bacterial consortia were also of Texas origin. Fumonisin levels in inoculations involving only the toxigenic Fusarium resulted in an averaged total fumonisin level of 36.5 ppm. The second study was designed to evaluate two additional scenarios besides the simultaneous inoculations of the corn ears with mixtures of the toxigenic fungus and each of five selected bacteria consortia, separately. The second scenario aimed to simulate the prophylactic application of each bacterial consortia to the corn ears as protection against a future infestation of the ears with a toxigenic Fusarium species. The third scenario aimed to simulate application of the respective bacterial consortia to corn ears post-infection by a toxigenic Fusarium species. The goal of the additional treatments was to expand the scope of evaluation of the potential of each bacterial consortia for the management of fumonisin issues in corn.

Results of the second study showed that although all of the selected bacterial consortia were effective in mitigating fumonisin accumulation in corn ears infected by the toxigenic Fusarium isolate, relative to inoculations involving just the fungus alone, four of the six consortia were consistently effective in all three scenarios studied (FIG. 3B). Applications of Texas bacterial consortia #9 post-infection of corn ears with the toxigenic Fusarium failed to achieve fumonisin levels below 4 ppm in the second study. This is in contrast to the same bacterial consortia (Texas #9) successfully achieving fumonisin levels below 4 ppm in the other two scenarios evaluated in the same study as well as in the first study. It is possible that the different environmental conditions under which the two studies were performed contributed to the varying results observed. However, the variation in study conditions also offered an opportunity to evaluate the effectiveness of the bacterial consortia over the same range of variations, thus helping to increase confidence in selections based on the multiple, rather than a single scenario. The results of the second study therefore suggests that the bacterial consortia—Texas-4, Texas-10, Texas-12, Texas-18, and Kansas-33 could potentially help to mitigate fumonisin accumulation in corn ears when applied prophylactically, simultaneously, or post-infection of ears with a toxigenic Fusarium pathogen.

Evaluation of the visual quality of the resultant kernels from the different treatments in the second study (FIG. 3C) showed consistencies with the result of the fumonisin quantifications from each of the treatments. Representative kernels as well as visual quality ratings, based on a 1-10 score, where 1 means less than ten percent of kernels with satisfactory visual quality and 10 equals hundred percent of kernels with satisfactory visual quality, is provided in FIG. 3C for the different treatments for the three scenarios evaluated in the second study.

Applications of the bacterial consortia post-inoculation with the toxigenic fungus resulted in fumonisin levels below 4 ppm, however, did not result in visual quality ratings above 7. Texas consortia #4 had the highest kernel visual quality rating (7) for the simultaneous inoculations of both the bacterial consortia and the fungus, while the Kansas consortia #18 had the highest visual quality rating (9) for inoculations of the bacterial consortia 96 h prior to inoculation with the fungus, that is B+F. Overall, however, the Texas consortia #4 had the highest combined rating for fumonisin mitigation in both studies as well as for kernel visual quality. The results indicated that further evaluations of Texas bacterial consortia numbers 4 and 10 and the Kansas bacterial consortia numbers 18 and 33 were warranted.

### Example 3

Whole-genome DNA as well as RNA transcripts from the candidate bacteria consortia identified in Examples 1 and 2 as showing antifungal phenotype were generated and sequenced using a combination of long- and short-read (genomic DNA) and short-read sequencing (RNA-seq) technologies. Bioinformatics analysis of the resultant sequence data was subsequently carried out resulting in the successful assembly and annotation of five bacteria genomes along with their respective analyzed transcriptomes under the evaluated experimental conditions of the current study which resulted in the identification differentially expressed candidate genes of interest.

The first objective was to conduct two independent studies evaluating the potential of the select candidate bacteria consortia to cause a reduction in the detectable final levels of introduced fumonisin of a known concentration in-vitro following direct exposure. Two bacterial consortia were identified that resulted in an average fumonisin level of 0.33 ppm and 0.58 ppm compared to the highest fumonisin level of >140 ppm measured in the study.

The second objective was to evaluate the gene expression profile, by long- and short-read RNA-Seq approaches, of bacteria consortia that results in a significant reduction in the detectable levels of fumonisin from the original introduced levels to identify and validate previously identified candidate genes potentially associated with the reduction. A further objective was to perform whole-genome sequencing, genome assembly and annotation of associated bacteria for the purpose of characterization given that none have previously been associated with fumonisin reduction potential, and thus represent a novel phenotype by this bacteria.

The genomes of five candidate bacteria associated with fumonisin-level reduction phenotypes were successfully assembled and annotated. The assembled genomes were used in the annotation of differentially expressed bacterial transcripts following direct exposure to fumonisin in-vitro. A total of forty-eight gene classes, based on the GO categories of cellular components, biological process, and molecular functions, were identified to be upregulated under the experimental conditions.

The detoxification of fumonisin (FB1) requires two important steps, a de esterification step resulting in the hydrolysis of fumonisin, followed by the deamination of the hydrolyzed fumonisin. Two microbial gene classes that can perform these important steps are hydrolases and aminotransferases.

In the current study, genes encoding for de esterification and deamination were identified among the differentially expressed genes by the bacteria evaluated. It is also noteworthy that the majority of the upregulated genes mapped to the genome of the Serratia sp. identified in the earlier stages of this work, showing potential for both inhibition of the mycotoxigenic Fusarium proliferatum isolate evaluated in this project as well as mitigation of fumonisin accumulation under the various experimental conditions that have been evaluated in the course of this project. Furthermore, genes for protein secretion as well as carboxylic acid and macromolecule catabolic processes were additionally identified among the differentially expressed genes of the Serratia sp. evaluated in this study.

The identified genes in this study are consistent with gene products known to be essential for the detoxification steps of fumonisin, especially fumonisin B1 and validates in particular the Serratia sp. as a candidate for mitigation of fumonisin accumulation in Fusarium-infected field corn.

Unlike previous studies that aimed to develop approaches for reducing fumonisin levels in contaminated corn products or mitigation of the adverse effects of consumption of fumonisins, the results from this current study identified candidate genes that can potentially function in the production of fumonisin detoxifying enzymes as well as, and importantly, the secretion of the enzymes to target fumonisin following their expression in field corn ears. The successful identification of previously validated gene classes in this study paves the way for the next phase involving the development and evaluation of functional gene constructs for the production of fusion products that can successfully detoxify fumonisins in-planta.

### Example 4

Results of the analysis of RNA transcripts were processed for selection of candidate gene candidates for inclusion in the generation of an initial set of eight fusion gene constructs. Due to large final sizes (construct sequence lengths) of two of the fusion gene constructs, of greater than 5 kilobases, only four were synthesized for transient expression and evaluation for anti-fumonisin activity of the respective fusion proteins.

Two vector systems incorporating an identical set of four synthetic fusion gene construct were developed and evaluated for production of their respective inserts designated as ‘FumMet’. The fusion protein of FumMet produced by the two-vector system was subsequently evaluated against purified fumonisin B1 for potential to metabolize the mycotoxin. Experimental protocols and results from repeated tests for FB1 levels were evaluated. The testing results showed that detectable FB1 in solutions exposed to the fusion protein products of the two vectors were consistently lower compared with the levels detectable in control setup (Table 2), indicating that the fusion genes construct have potential to metabolize FB1.

TABLE 2
Fumonisin B1 reduction by products of the fusion gene construct
evaluated in cell lysate, cell suspension, and cell culture supernatant,
respectively, of transformed Clean Genome ® ΔrecA E. coli.

| | Acetonitrile:water | | | DMSO | |
|---|---|---|---|---|---|
| | Experiment 1 | | Experiment 2 | Experiment 3 | |
| | Fumonisin (reduction) (ppm)* | | | | |
| Treatments | 3 HPI | | 20 HPI | 18 HPI | |
| Lysed cell culture | 18.7 | | — | 79.0 | (1.0) |
| Non-lysed cell culture | 10.4 | (3.6) | — | — | |
| Non-lysed cell culture supernatant | 10.7 | (3.3) | 15.0 (4.7) | 37.0 | (43) |

| | | | |
|---|---|---|---|
| Control (lysed cells) | 14 | — | — |
| Control (non-lysed cell culture supernatant) | 14 | 19.7 | 80 |

*Values represent the mean of three replicates.
Values in parenthesis represent amounts of FB1 metabolized by FumMet under the respective experimental conditions.
HPI-Hours post-incubation of fumonisin B1 with fusion enzymes.

The results of the evaluation of the fusion gene constructs further showed that FumMet is capable of a higher level of fumonisin metabolism (around 40 ppm, Table 2) compared with the reduction level of around 5 ppm in the acetonitrile-associated experiments.

A crucial aspect of the demonstration of the functionality of this technology was the selection of host cell for use in the evaluation the gene fusion construct. Clean Genome® ΔrecA E. coli cells (commercially available through Scarab Genomics, LLC (Madison, WI, USA)) were selected for this purpose. Clean Genome® ΔrecA E. coli cells are engineered to result in a higher level of recombinant protein expression and fewer host cell proteins. They are completely free of IS elements and cryptic prophages, plus deleted for many nonessential genes, thus providing enhanced genetic stability and improved metabolic efficiency, to maximize protein and nucleic production efficiency. Clean Genome® ΔrecA is an enhanced strain that is also recombination deficient, suitable for production of nucleic acids with structures that may be unstable in recA+E. coli, such as direct repeats, inverted repeats, stem-loops. They also feature custom protease gene deletions to allow production of proteins of interests in intact form.

Evaluation of the potential of FumMet to metabolize FB1 under different experimental conditions showed FunMet metabolized FB1 at a rate (˜4 ppm/˜24 hours) that is comparable to those of existing fumonisin detoxifying commercial products such as FUMDI. These commercial products employ an enzymatic mechanism that is different from the one utilized by FumMet (Table 2). Additionally, experimental data also suggests that FumMet has a significantly higher potential for metabolizing FB1 (˜40 ppm/˜20 hours) than any existing technology, further suggesting that the enzymatic pathway utilized by FumMet as superior to those of existing similar technologies (Table 2).

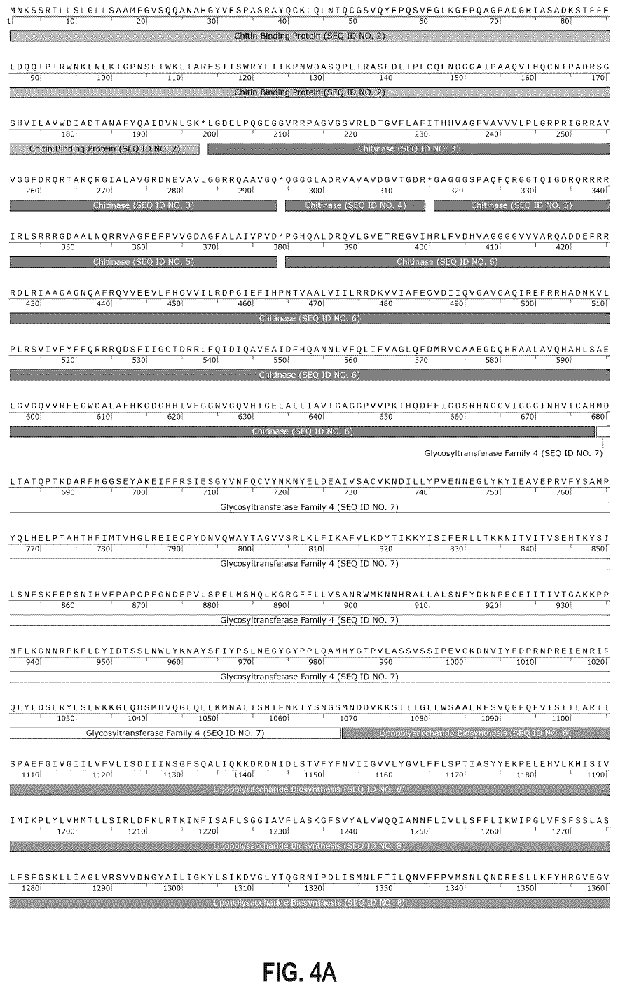

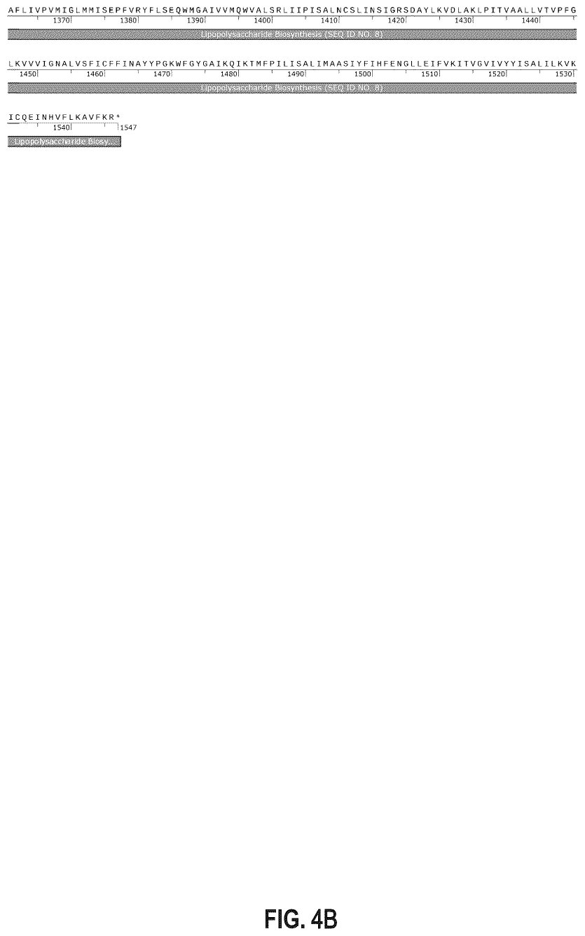

The sequence of FumMet is provided in SEQ ID NO: 1 and a map of the fusion protein is shown in FIGS. 4A and 4B. The construct features a glycosyltransferase family 4 protein fusion with a lipopolysaccharide biosynthesis protein as well as a chitinase and chitin-binding protein, which is involved in the breakdown of macromolecules.

Without being limited by theory, it is believed that the combined activities of aminotransferase and carboxylesterase enzyme activities of previously described fumonisin degrading enzymes are substituted by the combined activities of three genes whose enzymes result in (1) glycosyltransferase, and (2) macromolecule catalase activities on fumonisin B1 in two separate reactions. In the first reaction, the glycosyltransferase activity results in the catalysis of the transfer of a glycosyl group (deprotonation) from FB1 (donor) to lipid (acceptor), resulting in the protonation (hydrolysis) of the lipid molecule (acceptor), leading to the formation of glycolipids. The transfer of a glycosyl group is also thought to result in the formation of glycosidic linkages between the unit molecules of FB1. In the second reaction, the high relative molecular mass macromolecule, FB1, comprised of repeating units is thought to be broken down to component molecules of lower relative molecular masses. This predicted mechanism of fumonisin B1 metabolism by the fusion gene products described herein is distinct from those by other enzymes capable of metabolizing fumonisin B1, and thus constitutes a novel enzymatic mechanism of fumonisin metabolism (degradation) involving genes previously not associated with this process.

1. A plant variety having an ability to detoxify fumonisin, wherein a plant, a plant part, or a seed of said variety comprises a heterologous polynucleotide construct encoding a chitin-binding polypeptide, a chitinase polypeptide, a glycosyltransferase polypeptide, and a lipopolysaccharide biosynthesis polypeptide, wherein the chitin-binding polypeptide, chitinase polypeptide, glycosyltransferase polypeptide, and lipopolysaccharide biosynthesis polypeptide are Serratia sp. polypeptides.

2. The plant variety of claim 1, wherein the Serratia sp. comprises Serratia sp. isolate B1072.

3. The plant variety of claim 1, wherein the heterologous polynucleotide has at least 70%, at least 80%, at least 90%, at least 95%, at least 98%, at least 99%, or 100% sequence identity to SEQ ID NO: 1.

4. The plant variety of claim 1, wherein the chitin-binding polypeptide comprises an amino acid sequence having at least 70%, at least 80%, at least 90%, at least 95%, at least 98%, at least 99%, or 100% sequence identity to SEQ ID NO: 2.

5. The plant variety of claim 1, wherein the chitinase polypeptide comprises an amino acid sequence having at least 70%, at least 80%, at least 90%, at least 95%, at least 98%, at least 99%, or 100% sequence identity to SEQ ID NO: 3, 4, 5, and/or 6.

6. The plant variety of claim 1, wherein the glycosyltransferase polypeptide comprises an amino acid sequence having at least 70%, at least 80%, at least 90%, at least 95%, at least 98%, at least 99%, or 100% sequence identity to SEQ ID NO: 7.

7. The plant variety of claim 1, wherein the lipopolysaccharide biosynthesis polypeptide comprises an amino acid sequence having at least 70%, at least 80%, at least 90%, at least 95%, at least 98%, at least 99%, or 100% sequence identity to SEQ ID NO: 8.

8. The plant variety of claim 1, wherein the polynucleotide is operably linked to a heterologous promoter functional in a plant cell.

9. The plant variety of claim 1, wherein the fumonisin is fumonisin B1.

10. (canceled)

11. The plant variety of claim 1, wherein the plant is a maize, rice, wheat, barley, rye, sorghum, soybean, oat, or millet plant.

12. The plant variety of claim 1, wherein the plant further comprises a herbicide tolerance trait and/or a second fungal-resistant trait.

13. The plant variety of claim 1, wherein the plant variety comprises elite germplasm.

14. A plant, a plant part, a seed, or a plant cell of the plant variety of claim 1.

15. A method for controlling fumonisin contamination at a plant cultivation site, the method comprising:
providing at the site a plant of claim 14.

16. A commodity plant product prepared from the plant, plant part, plant seed, or plant cell of claim 14.

17. The commodity plant product of claim 16, wherein the product comprises fodder, seed meal, oil, milk, flour, grits, protein, fiber, tofu, tempeh, miso, starch, bio-composite building materials or seed-treatment-coated seed.

18. A method for producing a commodity plant product, the method comprising processing the plant, plant part, plant seed, or plant cell of claim 14 to obtain the product.

19. A method for producing a seed, the method comprising: (a) crossing a first plant with a second plant of the same species and (b) harvesting the resultant seed, wherein said first plant and/or second plant is a plant of claim 14.

20. A method of vegetatively propagating a plant of claim 14, the method comprising: (a) collecting a part capable of being propagated from the plant of claim 14 and (b) regenerating a plant from said part.

21. A method for producing a plant, plant part, or plant cell with an ability to detoxify fumonisin, the method comprising:
modifying the plant to comprise a heterologous polynucleotide construct encoding a chitin-binding polypeptide, a chitinase polypeptide, a glycosyltransferase polypeptide, and a lipopolysaccharide biosynthesis polypeptide, wherein the chitin-binding protein polypeptide, chitinase polypeptide, glycosyltransferase protein polypeptide, and lipopolysaccharide biosynthesis protein polypeptide are Serratia sp. polypeptides.

22. The method of claim 21, wherein the method comprises introducing a genome editing system into the plant, plant part, or plant cell.

23-29. (canceled)

30. A polynucleotide construct that confers fumonisin detoxification in plants, encoding a chitin-binding polypeptide, a chitinase polypeptide, a glycosyltransferase polypeptide, and a lipopolysaccharide biosynthesis polypeptide, wherein the chitin-binding polypeptide, chitinase polypeptide, glycosyltransferase polypeptide, and lipopolysaccharide biosynthesis polypeptide are Serratia sp. polypeptides.

31-35. (canceled)

36. The polynucleotide construct of claim 30, wherein the polynucleotide is operably linked to a heterologous promoter functional in a plant cell.

37. The polynucleotide construct of claim 30, wherein the polynucleotide construct encodes a fusion protein comprising the chitin-binding polypeptide, chitinase polypeptide, glycosyltransferase polypeptide, and/or lipopolysaccharide biosynthesis polypeptide.

38. A vector comprising the polynucleotide construct of claim 30.

39. A biological sample comprising the polynucleotide construct of claim 30.

40. A cell comprising the polynucleotide construct of claim 30.

41-43. (canceled)

44. A plant, plant part, plant seed, or plant cell comprising the polynucleotide construct of claim 30.

45. An antifungal composition comprising the polynucleotide construct of claim 30.

46. A method for controlling fumonisin contamination at a plant cultivation site, the method comprising:
contacting a plant at the plant cultivation site with an antifungal composition of claim 45.

47-52. (canceled)
